(19) United States
(12) Patent Application Publication
(10) Pub. No.: US 2004/0247625 A1
(43) Pub. Date: Dec. 9, 2004

(54) Pharmaceutical composition comprising a water/oil/water double microemulsion incorporated in a solid support

(76) Inventors: Fabio Carli, Trieste (IT); Elisabetta Chiellini, Pisa (IT)

Correspondence Address: STETINA BRUNDA GARRED & BRUCKER, 75 ENTERPRISE, SUITE 250, ALISO VIEJO, CA, 92656, US

(21) Appl. No.: 10/486,317

(22) PCT Filed: Aug. 8, 2002
(86) PCT No.: PCT/IB02/03172

(30) Foreign Application Priority Data
Aug. 9, 2001 (IT) MI01 A 001756

(51) Int. Cl. A61K 38/28; A61K 38/22; A61K 38/21; A61K 31/727; A61K 9/00

(52) U.S. Cl. 424/400; 514/12; 514/34; 514/56; 424/85.4; 514/9; 514/50; 514/3

(57) ABSTRACT

Pharmaceutical composition comprising a water/oil/water double microemulsion incorporated in a solid support consisiting of a microporous inorganic substance or of a colloidal inorganic adsorbent substance or of a cross-linked polymer. The liquid phase, i.e. the microemulsion, is incorported in the pores of a microporous inorganic substance or adsorbed by the surface of a colloidal inorganic substance or included in the cross-link of a cross-linked polymer.

### PRIOR ART

As described in French patent No. 8602694, water/oil (w/o) single emulsions containing a drug are transformed into “solid emulsions” through adsorption of same by a hydrophilic-hydrophobic silica mixture. The resulting water-soluble drugs are slow-releasing type and, therefore, may be potentially used as controlled release forms (A. Berthod et al., J.Pharm. Sci., 77, 216-221, 1988).

Water/oil (w/o) or oil/water (o/w) single microemulsions loaded on adsorbent powders are described e.g. in patent WO 00/09093. Out of their many applications, said microemulsions are suitable for the oral administration of drugs that—due to their polarity—can be little absorbed as they are scarcely permeable through the gastrointestinal mucous membrane.

Water/oil (w/o) single microemulsions, aimed at increasing the oral absorption of little permeable drugs, are also described in patent WO 9003164. In this case, the liquid microemulsion is formulated in the solid form as it is loaded on polymers and granulated.

However, said solid emulsions and microemulsions exhibit some unsatisfactory features. By way of example, their permeability through the gastrointestinal mucous membrane increases but is still insufficient for most drugs. Furthermore, the drug cannot be loaded in large amounts on the solid support. It follows that a considerable active ingredient dilution is needed, which arouses problems in terms of size and volume of the final pharmaceutical form.

### SUMMARY OF THE INVENTION

It has surprisingly been found that the problems encountered in the prior art are solved by using the pharmaceutical composition according to the present invention.

Said pharmaceutical composition comprises a water/oil/water (w/o/w) double microemulsion incorporated in a solid support. The internal aqueous phase of said microemulsion contains a dissolved drug.

The preferred drugs according to the present invention are the drugs exhibiting low permeability through the gastrointestinal mucous membrane, more preferably the drugs having a polypeptidic/proteic structure and those having a high polarity or a high molecular weight.

The solid support consists of a microporous inorganic substance or of a colloidal inorganic adsorbent substance or of a cross-linked polymer.

With said composition, the gastrointestinal mucous membrane is extremely permeable to the drug; furthermore, the drug can be loaded in high amounts on the solid support.

### BRIEF DESCRIPTION OF THE FIGURES

FIG. 1 shows the w/o/w microemulsion adsorption index by silica as per Example. 1. The grams of the adsorbed w/o/w microemulsion per 100 g silica are plotted on the ordinate axis.

FIG. 2 and FIG. 3 have the same meaning as FIG. 1, referred to Examples 3 and 4, respectively.

FIG. 4 shows the adsorption index of the w/o/w microemulsion by silica as per Example 5.

FIG. 5 shows the adsorption index of the w/o/w microemulsion by silica as per Example 3 (curve a) in comparison with the adsorption index of the w/o microemulsion (curve b) by the same type of silica.

FIG. 6 represents the size of the oily microdrops released from the compositions of Example 1 (curve a) and of Example 3 (curve b), in a buffer solution, pH 7.4, at 37° C.

FIG. 7 shows the permeability ex vivo on the rat of acyclovir from the composition of Example 1 (curve a) in comparison with acyclovir as is (curve b).

FIG. 8 shows the permeability ex vivo on the rat of acyclovir from the w/o composition of Example 3 (curve a) in comparison with a w/o composition incorporated in the same type of silica (curve b).

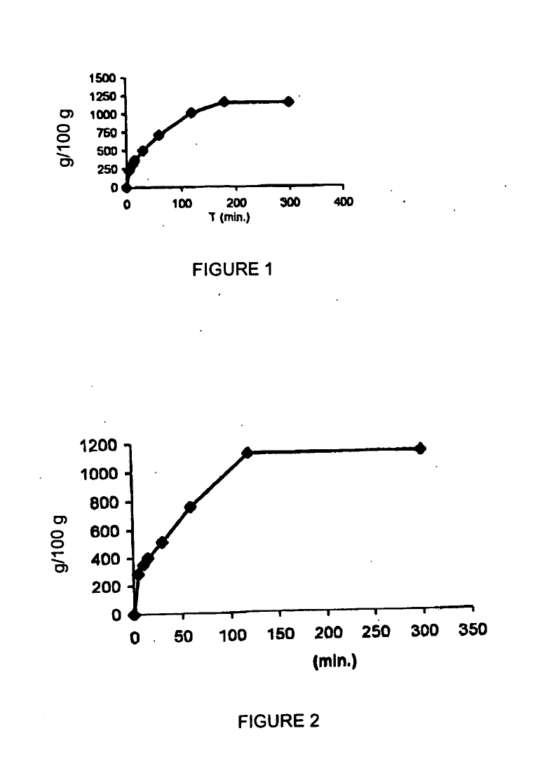

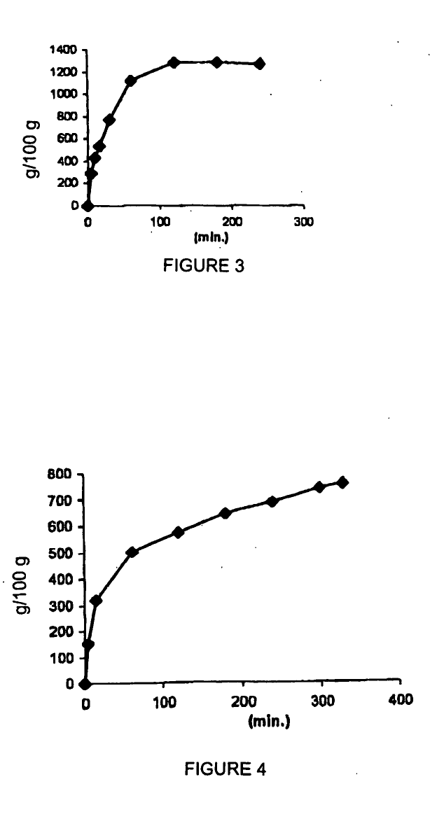

### DETAILED DESCRIPTION OF THE INVENTION

The characteristics and advantages of the pharmaceutical composition comprising a water/oil/water double microemulsion incorporated in a solid support according to the present invention will be apparent from the detailed description that follows. The microemulsion of the invention may be prepared according to a procedure consisting in the following steps:

a) drug dissolution in water;

b) addition of the solution as per a) to an oil;

c) addition of a surfactant/cosurfactant to the mixture as per b) and stirring with formation of the w/o microemulsion;

d) addition of the microemulsion as per c) to a water/surfactant/cosurfactant mixture and stirring with formation of the who/w double microemulsion.

The resulting w/o/w microemulsion is incorporated in the solid support to give the composition according to the present invention.

Minor modifications to this process may be effected, while the composition characteristics are kept constant.

As may be inferred from the process description, the who/w double microemulsion consists of water, oil, surfactant and cosurfactant, and the internal aqueous phase contains a dissolved drug.

Said double microemulsion shows the following composition by weight of the aqueous and oily phases:

| | |
|---|---|
| Water in the internal phase (w/o) | 0.5 to 5.0% |
| Oil | 3.0 to 10.0% |
| Surfactant | 2.0 to 15% |
| Cosurfactant (w/o internal phase) | 0 to 2.0% |
| Cosurfactant (w/o/w external phase): | 1.0 to 10% |
| Water in the external phase (w/o/w) | 50 to 90% |

Preferred drugs according to the present invention are the drugs soluble in water and poorly permeable through the gastrointestinal mucous membrane, in particular the drugs having a proteic/polypeptidic structure and those having a high polarity and a high molecular weight.

Said drugs are e.g. calcitonine, heparin, acyclovir, antibiotics, such as cephalosporins, insulin, interpheron, erythropoietin, doxorubicin, adriamycin, growth hormone, and somatotropin.

The w/o microemulsion aqueous phase, characterised in that the drug is dissolved, may be deionised water or a buffer solution at different pH values (1; 2; 5.5 and 7.5).

The microemulsion oily phase may be an oil of vegetable origin, such as olive oil, soya bean oil, maize oil, coconut oil, or of synthetic origin, such as esters of fatty acids, e.g. isopropylmiristate, isopropylpalmitate. Fatty acids may have a short or medium or long chain.

Also mixtures of mono-, di- and triglycerides of vegetable or synthetic or semi-synthetic origin or polyethoxylated derivatives thereof may be used.

The surfactants used in the microemulsion must be non-toxic, of synthetic or of natural origin. Surfactant combinations having different chemical characteristics may be also used.

The surfactants particularly suitable for the invention are Tween®, Brij®, Cremophor®, Span®, Myverol®.

The surfactants used in the preparation of the w/o/w microemulsion may be of synthetic origin, such as for example short-chain alcohols, e.g. ethanol, isopropanol, or of natural origin, such as for example lecithins, phospholipids or derivatives thereof.

The solid support consists of microporous inorganic substances or of high-surface-area colloidal inorganic adsorbent substances or of cross-linked polymers.

The microporous inorganic substances are selected from the group consisting e.g. of silica, silicates, zeolites, aluminas, activated carbons.

The adsorbent colloidal inorganic substances are selected from the group consisting e.g. of colloidal silica, magnesium trisilicate, clays, magnesium hydroxide, talc.

The cross-linked polymers are selected from the group consisting of crospovidone, cross-linked sodium carboxymethyl starch, cross-linked sodium carboxymethyl cellulose, cross-linked polystyrene, cross-linked polymethyl methacrylate.

In the w/o microemulsion the water/oil ratio ranges from 0.7 to 0.2 by weight.

In the w/o microemulsion the water/surfactant ratio ranges from 0.50 to 1.5 by weight.

In the water/surfactant/cosurfactant mixture used in d), the water/surfactant ratio ranges from 8:1 to 12:1 by weight.

In the water/surfactant/cosurfactant mixture used in d), the water/cosurfactant ratio ranges from 9:1 to 15:1 by weight.

The ratio of the water/surfactant/cosurfactant mixture to the w/o microemulsion used in d) ranges from 8:1 to 13:1 by weight.

The ratio of the w/o/w double microemulsion to the solid support ranges from 1:10 to 15:1 by weight.

The drug content in the final composition ranges from 1 ppm to 50% by weight.

The w/o microemulsion is prepared at a temperature of 20° C. to 65 ° C.

The w/o/w double microemulsion is prepared at a temperature of 20° C. to 55° C.

The incorporation of the w/o/w double microemulsion is performed at a temperature of 20° C. to 55° C., by means of high-efficiency apparatus, such as mixers, granulators and extruders.

The resulting composition is in the form of free-flowing powder having 0.1 to 800 μm particle size or in the form of a plastic mass workable by extrusion and spheronising, or by addition of further suitable excipients.

The compositions according to the present invention exhibit some important features:

1. The oily microdrops released from the solid support into an aqueous medium are less than 1 micrometer in size.

2. The drug dissolution rate in an appropriate aqueous buffer can be adjusted from a few-minute immediate release to a some-hour delayed release.

3. The permeability of little permeable drugs through the gastrointestinal mucous membrane increases considerably.

Said compositions for therapeutic use are prepared in the form of capsules or sachets or tablets or suspensions containing a pharmaceutically effective amount of drug and pharmaceutically acceptable excipients or diluents, if any.

The following examples referred to the preparation and characterisation of the claimed compositions are conveyed to illustrate the present invention.

### EXAMPLE 1

| Double microemulsion (w/o/w) components | Quantity (g) |
|---|---|
| Isopropylmiristate | 0.846 |
| Lecithin Lipoid S-40 ® | 0.282 |
| Tween 80 | 2.30 |
| Acyclovir | 0.005 |
| Buffer, pH 1.4 | 0.517 |
| Water | 23.1 |
| Ethanol | 2.31 |

A w/o microemulsion was prepared by mixing, under magnetic stirring at 25° C., an isopropylmiristate/lecithin solution (3:1) as an oily phase, a buffer solution, pH 1.4, containing dissolved acyclovir as an aqueous phase (0.517 g), and surfactant Tween 80 (0.8 g).

The w/o microemulsion was added under stirring (600 rpm for 100 min) to a water/ethanol/Tween 80 mixture (15:1.5:1.0) in a microemulsion/(water/ethanol/Tween 80) ratio of 0.09:1 to give the w/o/w double microemulsion containing acyclovir.

The resulting w/o/w double microemulsion was incorporated in colloidal silica Aerosil 300® by granulation in a high-efficiency granulator (Battagion, Bergamo, Italy).

The ratio of the w/o/w microemulsion to silica was equal to 7.6:1 by weight.

The composition obtained was in the form of free-flowing powder having a uniform particle size.

### EXAMPLE 2

| Double microemulsion (w/o/w) components | Quantity (g) | |
|---|---|---|
| Isopropylmiristate | 1.66 | |
| Lecithin Lipoid S-40 ® | 0.550 | |
| Sodium heparin | 5000 | I.U. |
| Water (internal phase) | 1.034 | |
| Tween 80 | 4.51 | |
| Water (external phase) | 46.2 | |
| Ethanol | 4.53 | |

A w/o microemulsion was prepared by mixing at 25° C. an isopropylmiristate/lecithin solution (3:1) as an oily phase, water (1.034 g) containing heparin, and surfactant Tween 80 (1.5 g).

The mixture was maintained under magnetic stirring at 600 rpm for 1 hr.

The w/o microemulsion was added to a water/ethanol/Tween 80 mixture (15:1.5:1.0) in a microemulsion/(water/ethanol/Tween 80) ratio of 0.09:1.

The mixture was maintained under magnetic stirring at 600 rpm for 1 hr to give the w/o/w double microemulsion.

The resulting w/o/w double microemulsion was incorporated in cross-linked crospovidone polymer, Kollidon CL®, in a twin screw extruder APV. Finally the product was spheronised.

The ratio of the w/o/w microemulsion to crospovidone was equal to 1:1 by weight.

The free-flowing powder obtained had a uniform particle size.

### EXAMPLE 3

| Double microemulsion (w/o/w) components | Quantity (g) |
|---|---|
| Akoline ® | 0.672 |
| Acyclovir | 0.127 |
| Water | 13.9 |
| Tween 80 | 0.470 |
| Buffer, pH 1.4 | 0.277 |

A w/o microemulsion was prepared by mixing, under magnetic stirring at 35°C., Akoline® as an oily phase, a buffer solution, pH 1.4, containing dissolved acyclovir as an aqueous phase (0.227 g), and surfactant Tween 80 (0.470 g).

The w/o microemulsion was added under stirring (600 rpm for 60 min) to a water/ethanol mixture (10:1.0) in a (water/ethanol)/microemulsion ratio of 9.9:1 to give a w/o/w double microemulsion containing acyclovir.

The resulting w/o/w double microemulsion was incorporated in colloidal silica in a 7.7:1 ratio.

The composition obtained was in the form of free-flowing powder having a uniform particle size.

### EXAMPLE 4

| Double microemulsion (w/o/w) components | Quantity | |
|---|---|---|
| Labrasol ® | 1.60 | g |
| Tween 80 | 1.90 | g |
| Calcitonine | 2200 | I.U. |
| Acid aqueous solution | 41.3 | g |
| Water | 41.3 | g |
| Ethanol | 4.1 | g |

A w/o microemulsion was prepared at 25° C. by addition of Labrasol/Tween 80 solution (2.9:1) to the 0.1 N HCl acid aqueous solution containing calcitonine (2200 I.U./ml).

The mixture was maintained under magnetic stirring at 600 rpm for 40 min, added with the remainder of the Tween 80, and allowed to stir at 600 rpm for 1 hr.

The resulting w/o microemulsion was added to a water/Tween 80/ethanol solution in a microemulsion/(water/Tween 80/ethanol) ratio of 0.101:1. The mixture was allowed to stir at 450 rpm for 30 min, and then at 500 rpm for 60 min. A w/o/w microemulsion was obtained.

The microemulsion was incorporated in colloidal silica by means of a high-efficiency granulator, in an emulsion/colloidal silica ratio of 12.5:1 by weight.

### EXAMPLE 5

| Double microemulsion (w/o/w) components | Quantity (g) |
|---|---|
| Isopropylmiristate | 0.846 |
| Lecithin Lipoid S-40 ® | 0.282 |
| Tween 80 | 2.30 |
| Acyclovir | 0.005 |
| Buffer, pH 1.4 | 0.517 |
| Water | 23.1 |
| Ethanol | 2.31 |

A w/o microemulsion was prepared by mixing, under magnetic stirring at 25° C., an isopropylmiristate/lecithin solution (3:1) as an oily phase, a buffer solution, pH 1.4, containing dissolved acyclovir as an aqueous phase (0.517 g), and surfactant Tween 80 (0.8 g).

The w/o microemulsion was added under stirring (600 rpm for 100 min) to a water/ethanol/Tween 80 mixture (15:1.5:1.0) in a microemulsion/(water/ethanol/Tween 80) ratio of 0.09:1 to give the w/o/w double microemulsion containing acyclovir.

The w/o/w double microemulsion obtained was incorporated in microporous silica Syloid® by granulation in a high-efficiency granulator (Battagion, Bergamo, Italy).

The ratio of the w/o/w microemulsion to silica was equal to 5.0:1 by weight.

The resulting composition was in the form of powder.

### Characterisation Tests

The compositions of the present invention were characterised by the following procedures.

Adsorption Index

The index of adsorption of the w/o/w microemulsion and the w/o microemulsion as per Example 1 by silica as per Example 1 was determined using a fritted bottom cylinder.

The amount of liquid adsorbed by silica was determined gravimetrically by the difference between the weight of the powder after adsorption and the weight of silica as is.

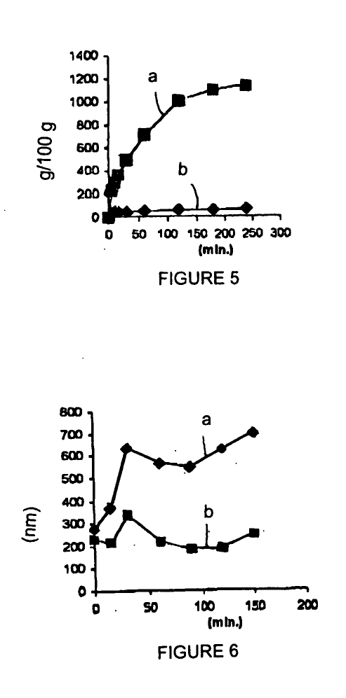

The adsorption index of the w/o/w double microemulsion is higher than that of the w/o microemulsion (FIG. 5).

The adsorption index of the w/o/w microemulsion by colloidal silica increases over

time and can vary depending on the type of the basic oily mixture used (FIG. Nos. 1, 2, and 3). The adsorption index of a double microemulsion by microporous silica is shown in FIG. 4.

Size of the Microdrops Released from the Compositions of the Invention

The size of the microdrops released from the compositions as per Examples 1 and 3 was determined by dispersing the composition under magnetic stirring in a buffer solution, pH 7.4, at 37° C. Samples of 4 ml each were taken from the suspension at 0, 15, 30, 60, 90, 120, 150 min intervals. After centrifuging, the supernatant was analysed by the Laser Light Scattering technique. The microdrops size ranged from 100 to 700 nm (FIG. 6).

In vitro Release Test

The amount of acyclovir released over time from the compositions as per Examples 1 and 5 was determined by suspending the composition in 900 ml buffer, pH 7.4, under stirring (100 rpm at 37° C.). At predetermined times, adequate samples were taken and centrifuged. The supernatant was analysed by HPLC.

TABLE 1
Acyclovir release rate from the compositions
as per Examples 1 and 5

| Time (hr) | % release-Example 1 | % release-Example 5 |
|---|---|---|
| 0.15 | 95.0 | 2.0 |
| 1.0 | 99.5 | 7.5 |
| 6.0 | 100.0 | 64.0 |

The data of Table 1 show that the release rates differ considerably depending on the types of silica used as a solid support (colloidal silica as per Example 1 and microporous silica as per Example 5).

Ex vivo Permeation Tests

The ex vivo permeation tests were performed on Wistar rats weighing about 250-300 g, fasted for approx. 8 hrs prior to the experiment. The animal was anaesthetized by ethyl urethane injected i.m. (1.5 mg/kg bodyweight).

The peritoneum was opened and the intestinal tract, from the Treitz ligament to approx. mid-jejunum, was isolated. The isolated intestinal tract was cannulated by two cannulas, for the perfusate inlet and outlet, respectively. Once the isolated intestinal tract was washed with a buffer solution, pH 7.4, it was perfused with a suspension containing the active ingredient in a concentration of 120 γ/ml of the composition as per Example 1, in a buffer, pH 7.4. The recycle method was used. Samples of 0.1 ml were taken from the mother suspension at various intervals (0, 15, 30, 60 min), centrifuged and analysed by HPLC.

The active ingredient absorption through the intestinal wall was calculated by the difference between the value of the initial concentration and the value of the recycling suspension at the various time intervals.

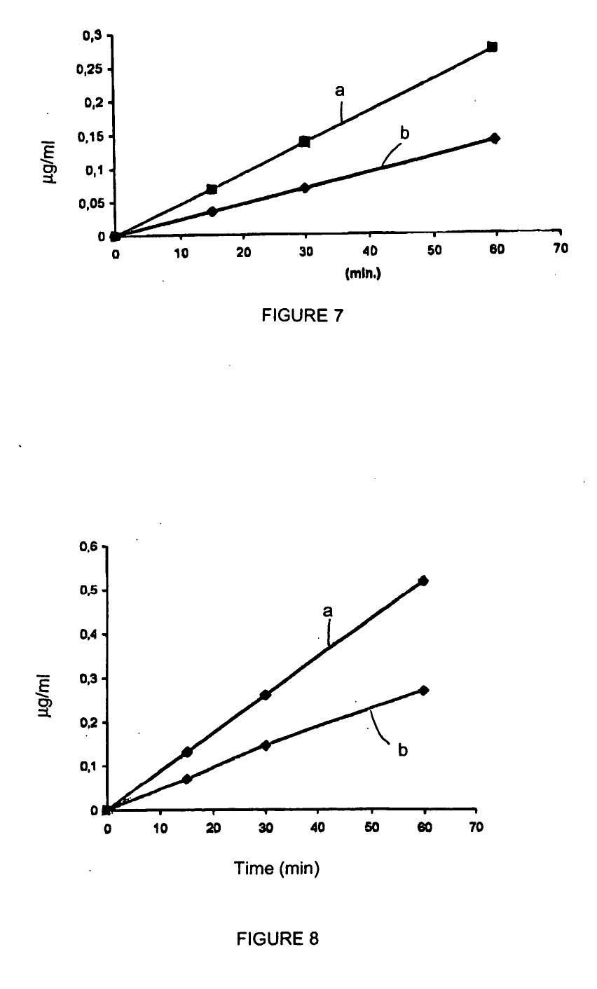

The permeability found in the case of the suspensions obtained with the w/o/w double microemulsion in colloidal silica is considerably higher than that of the active ingredient as is (FIG. 7).

Furthermore, the data shown in FIG. 8 prove that, using the same colloidal silica as a solid support, the permeability that may be obtained with a w/o/w double microemulsion according to the invention is much higher than that obtainable with a single w/o microemulsion.

1. Pharmaceutical composition comprising a water/oil/water double microemulsion whose aqueous phase contains a dissolved drug, characterised in that said double microemulsion is incorporated in a solid support selected from a microporous inorganic substance, a colloidal adsorbent substance and a cross-linked polymer.

2. The composition as claimed in claim 1, characterised in that said microporous inorganic substance is selected from the group consisting of silica, silicates, zeolites, aluminas and activated carbons.

3. The composition as claimed in claim 1, characterised in that said colloidal adsorbent substance selected from the group consisting of colloidal silica, magnesium trisilicate, clays, magnesium hydroxide, and talc.

4. The composition as claimed in claim 1, characterised in that said cross-linked polymer is selected from the group consisting of crospovidone, cross-linked sodium carboxymethyl starch, cross-linked sodium carboxymethyl cellulose, cross-linked polystyrene, cross-linked polymethyl methacrylate.

5. The composition as claimed in claim 1, characterised in that said double microemulsion consists of water (w/o internal phase) in a quantity of 0.5 to 5.0%, oil in a quantity of 3.0 to 10%, a surfactant in a quantity of 2.0 to 15%, and a cosurfactant (w/o internal phase) in a quantity of 0 to 2.0%, water (w/o/w external phase) in a quantity of 50 to 90%, a cosurfactant (w/o/w external phase) of 1.0 to 10%.

6. The composition as claimed in claim 1, characterised in that the ratio of said microemulsion to said solid support ranges from 1:10 to 15:1 by weight.

7. The composition as claimed in claim 1, characterised in that the drug content ranges from 1 ppm to 50.0%.

8. The composition as claimed in claim 1, characterised in that said drug is selected from the group comprising calcitonine, heparin, acyclovir, cephalosporins, insulin, interpheron, erythropoietin, doxorubicin, adriamycin, growth hormone, and somatotropin.

9. The composition as claimed in claim 5, characterised in that said oil is selected from the group comprising olive oil, soya bean oil, maize oil, coconut oil, isopropylmiristate, isopropylpalmitate, and mono-, di- and triglycerides mixtures or polyethoxylated derivatives thereof.

10. The composition as claimed in claim 5, characterised in that said surfactant is selected from the group consisting of Tween®, Brij®, Cremophor®, Span®, Myverol®.

11. The composition as claimed in claim 5, characterised in that said cosurfactant is selected from the group consisting of ethanol, isopropanol, lecithins and phospholipids or derivatives thereof.

12. Double microemulsion (w/o/w) containing a dissolved drug in the internal aqueous phase, having the following by wt. composition of the aqueous and oily phases:

| | |
|---|---|
| Water of the internal phase (w/o) | 0.5 to 5.0% |
| Oil | 3.0 to 10.0% |
| Surfactant | 2.0 to 15.0% |
| Cosurfactant (w/o internal phase) | 0 to 2.0% |
| Cosurfactant (w/o/w external | 1.0 to 10.0% |
| phase) | |
| Water of the external phase | 50 to 90.0% |
